(19) United States
(12) Patent Application Publication
(10) Pub. No.: US 2021/0085839 A1
(43) Pub. Date: Mar. 25, 2021

(54) NEGATIVE-PRESSURE THERAPY WITH OXYGEN

(71) Applicant: KCI Licensing, Inc., San Antonio, TX (US)

(72) Inventors: Justin Alexander LONG, Lago Vista, TX (US); Timothy Mark ROBINSON, Shillingstone (GB); Christopher Brian LOCKE, Bournemouth (GB)

(21) Appl. No.: 16/613,479

(22) PCT Filed: Jun. 8, 2018
(86) PCT No.: PCT/US2018/036740

§ 371 Date: Nov. 14, 2019

Related U.S. Application Data
(60) Provisional application No. 62/517,066, filed on Jun. 8, 2017.

(51) Int. Cl. A61M 1/00; A61M 35/00

(52) U.S. Cl. CPC A61M 1/0025; A61M 1/0088; A61M 1/0031; A61M 2202/0208; A61M 2205/3327; A61M 2205/3331; A61M 35/30

(57) ABSTRACT

Systems, apparatuses, and methods for providing negative-pressure therapy with oxygen therapy are claimed. An apparatus for providing negative pressure therapy with normobaric or hypobaric oxygen therapy may include dressing having a tissue interface and a cover. Additionally, in some embodiments, the apparatus may include a negative-pressure source and an oxygen-concentrating or oxygen-generating source, each of which may be coupled to or configured to be coupled to the tissue interface. The tissue interface can enable fluid transport during negative-pressure therapy cycles, and disbursement of normobaric or hyperbaric oxygen during oxygen therapy cycles. For example, some embodiments of the tissue interface may comprise structures or foams constructed from polyurethane, silicone, or polyvinyl chloride. The cover should be versatile enough to conform to a tissue site, yet be sufficiently inelastic or become sufficiently inelastic to contain and sustain the pressure from the application of topical normobaric oxygen therapy or hypobaric oxygen therapy.

### CROSS-REFERENCE TO RELATED APPLICATIONS

This application claims the priority benefit under 35 USC § 119(e) of U.S. Provisional Application No. 62/517,066, entitled “Negative-Pressure Therapy With Oxygen,” filed Jun. 8, 2017, which is incorporated herein by reference in its entirety.

### TECHNICAL FIELD

The invention set forth in the appended claims relates generally to tissue treatment systems and more particularly, but without limitation, to negative-pressure therapy with normobaric or hyperbaric oxygen therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

The application of concentrated oxygen to a tissue site can also be highly beneficial for new tissue growth or healing. For example, hyperbaric oxygen therapy may be particularly beneficial for tissue with poor oxygenation, such as often seen in diabetic foot ulcers.

While the clinical benefits of negative-pressure therapy and oxygen therapy are widely known, improvements to therapy systems, components, and processes may continue to benefit healthcare providers and patients.

### BRIEF SUMMARY

New and useful systems, apparatuses, and methods for providing negative-pressure therapy with oxygen therapy are set forth in the appended claims. Illustrative embodiments are also provided to enable a person skilled in the art to make and use the claimed subject matter.

For example, in some embodiments, an apparatus for providing negative pressure therapy with oxygen therapy may include a dressing having a tissue interface and a cover. Some embodiments may comprise or consist essentially of a dressing with an oxygen sensor. Additionally, in some embodiments, the apparatus may include a negative-pressure source and an oxygen-concentrating or oxygen-generating source, each of which may be coupled to or configured to be coupled to the tissue interface.

The tissue interface can enable fluid transport during negative-pressure therapy cycles, and disbursement of oxygen during oxygen therapy cycles. For example, some embodiments of the tissue interface may comprise structures or foams constructed from polyurethane, silicone, or polyvinyl chloride. Additionally or alternatively, the tissue interface may comprise a non-woven, such as a compressed Polyolefin or co-polyester. In some embodiments, the tissue interface may additionally or alternatively comprise a layer of perforated silicone gel adhesive.

The cover may provide a conformable and customizable seal around the tissue interface to contain topical oxygen, while also providing a sterile barrier to infection. The cover may comprise a carrier substrate or film, such as a polyurethane or polyethylene, and may be coated with an acrylic-based adhesive. In some embodiments, the cover is preferably adapted to withstand hyperbaric pressures of up to 3.0 atmospheres. The cover should be versatile enough to conform to a tissue site, yet be sufficiently rigid or become sufficiently rigid to contain and sustain the pressure from the application of topical normobaric oxygen therapy or hypobaric oxygen therapy. In some embodiments, for example, the cover may be comprised of any naturally stiff or stretch-resistant polyethylene substrate, film, or foam, or it may be constructed from Glyptal or pentaphthalic from the Alkyd family of medical grade substrates, films, or foam polymers that cross-link when exposed to oxygen at room temperature (approximately 20° C.). Alternatively, it may also be comprised of other polymers that cross-link upon exposure to body heat, exposure to carbon dioxide, or that evaporate a volatile plasticizer to stiffen, such as substrates, films, or foams made from polyvinyl alcohol.

The oxygen sensor may comprise or consist essentially of a chemical adapted to react to oxygen, for example, to indicate the presence or concentration of oxygen through a color change or other transformational change. The indicator may be a colorimetric response in some embodiments, and the dressing may further include a corresponding colorimetric scale.

In some apparatuses or systems, one or more tubes may fluidly couple the tissue interface to a negative-pressure source and an oxygen source. In some embodiments, a single multi-lumen tube may fluidly couple the tissue interface to the negative-pressure source and to the oxygen source. For example, the normobaric or hyperbaric oxygen can be dispensed through outer lumens of a multi-lumen tube, or through a separate single-lumen tube. The fluid connection may also be made from other flexible conduits, such as a foam or non-woven encapsulated in an occlusive film. Absorbents and super-absorbents such as polyacrylates may also be incorporated into some embodiments.

Any known type of negative-pressure source may be used, including without limitation vacuum pumps, wall suction, or a venturi with a positive-pressure source. The oxygen source may be an oxygen concentrator (active filtration), oxygen generator (electrolysis), oxygen storage canister, or wall oxygen source, for example. In some embodiments, a peristaltic pump may be used to meter or control oxygen. A valve may also be configured to switch between delivery of negative pressure and oxygen in some embodiments.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure therapy and oxygen therapy in accordance with this specification;

FIG. 2 is a schematic diagram illustrating additional details that may be associated with an example embodiment of the dressing of the therapy system of FIG. 1;

FIG. 3 is a simplified flow diagram illustrating additional details that may be associated with some example embodiments of the therapy system of FIG. 1;

FIG. 4 is a schematic diagram of an example embodiment of the dressing of the therapy system of FIG. 1 with a colorimetric oxygen-sensing indicator, and a corresponding colorimetric scale indicative of oxygen concentration; and

FIG. 5 is a schematic diagram of another example embodiment of the dressing of the therapy system of FIG. 1 with a colorimetric oxygen-sensing indicator, and a corresponding colorimetric scale.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

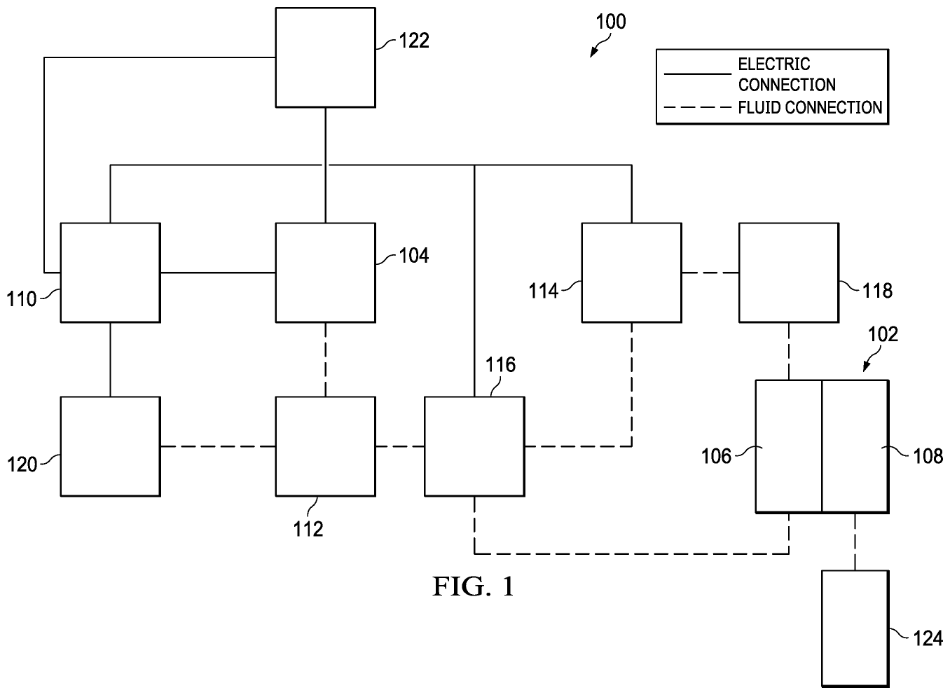

FIG. 1 is a simplified functional block diagram of an example embodiment of a therapy system 100 that can provide negative-pressure therapy and oxygen therapy to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a negative-pressure supply, and may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be a distribution component fluidly coupled to a negative-pressure source 104, as illustrated in FIG. 1. A dressing may include a cover, a tissue interface, or both in some embodiments. The dressing 102, for example, may include a cover 106 and a tissue interface 108. A regulator or a controller, such as a controller 110, may also be coupled to the negative-pressure source 104.

In some embodiments, a dressing interface may facilitate coupling the negative-pressure source 104 to the dressing 102. For example, such a dressing interface may be a T.R.A.C.® Pad or Sensa T.R.A.C.® Pad available from KCI of San Antonio, Tex. The therapy system 100 may optionally include a fluid container, such as a container 112, coupled to the dressing 102 and to the negative-pressure source 104.

The therapy system 100 may also include a source of oxygen. For example, an oxygen source 114 may be fluidly coupled to the dressing 102, as illustrated in the example embodiment of FIG. 1. A regulator, such as the regulator 118, may also be fluidly coupled to the oxygen source 114 and/or the dressing 102 to regulate oxygen delivered to or pressure in the dressing 102. In some embodiments, for example, the regulator 118 may be a pressure relief valve.

In some embodiments, a control valve 116 may also be fluidly coupled to the negative-pressure source 104 and to the oxygen source 114. The control valve 116 may also be coupled to the controller 110, which may be configured to switch the control valve 116 to alternately couple the dressing 102 to the negative-pressure source 104 and the oxygen source 114.

Additionally, the therapy system 100 may include sensors to measure operating parameters and provide feedback signals to the controller 110 indicative of the operating parameters. As illustrated in FIG. 1, for example, the therapy system 100 may include a pressure sensor 120, an electric sensor 122, or both, coupled to the controller 110. The pressure sensor 120 may also be coupled or configured to be coupled to a distribution component and to the negative-pressure source 104. Some embodiments of the therapy system 100 may also include an oxygen sensor 124. For example, the oxygen sensor 124 may be coupled to the dressing 102 as illustrated in the example of FIG. 1. In some embodiments, the oxygen sensor 124 may be integral to the dressing 102. For example, the oxygen sensor 124 may be disposed between the cover 106 and the tissue interface 108 in some embodiments, or may be applied, bound, or coated on the cover 106 or the tissue interface 108.

Components may be fluidly coupled to each other to provide a path for transferring fluids (i.e., liquid and/or gas) between the components. For example, components may be fluidly coupled through a fluid conductor, such as a tube. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. For example, a tube may mechanically and fluidly couple the dressing 102 to the container 112 in some embodiments.

In general, components of the therapy system 100 may be coupled directly or indirectly. For example, the negative-pressure source 104 may be directly coupled to the controller 110, and may be indirectly coupled to the dressing 102 through the container 112.

The fluid mechanics of using a negative-pressure source or an oxygen source to move fluid in a system can be mathematically complex. However, the basic principles of fluid mechanics applicable to negative-pressure therapy and oxygen therapy are generally well-known to those skilled in the art, and the process of reducing pressure or moving oxygen may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located, or approximated as standard atmospheric pressure at sea level. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mmHg (−667 Pa) and −500 mmHg (−66.7 kPa). Common therapeutic ranges are between −75 mmHg (−9.9 kPa) and −300 mmHg (−39.9 kPa).

Normobaric pressure generally refers to standard atmospheric pressure at sea level, or 1 atmosphere (atm). Hypobaric pressure generally refers to pressure less than normobaric pressure, and hyperbaric pressure generally refers to pressure greater than normobaric pressure. Therapeutic ranges of pressurized oxygen may vary. In some embodiments, the therapy system 100 may provide oxygen therapy at negative pressure up to 0.26 atmospheres (−200 mmHg or −26 kPa), normobaric pressure (101 kPa), hyperbaric pressure up to 3 atmospheres (304 kPa), or some combination thereof.

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. The oxygen source 114 may be an oxygen concentrator (active filtration), oxygen generator (electrolysis), oxygen storage canister, or wall oxygen source, for example. The oxygen source 114 may be capable of supplying oxygen at both a flow rate and back pressure required to achieve hyperbaric pressures. In some instances, the oxygen source 114 may include an over-pressure relief valve.

In some embodiments, the negative-pressure source 104 and the oxygen source 114 may be housed within or used in conjunction with other components, such as sensors, processing units, alarm indicators, memory, databases, software, display devices, or user interfaces that further facilitate therapy. For example, in some embodiments, the negative-pressure source 104 may be combined with the oxygen source 114, the controller 110, and other components into a therapy unit. One or more supply ports may also be configured to facilitate coupling and de-coupling the negative-pressure source 104 and the oxygen source 114 to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may comprise or consist essentially of a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as GranuFoam® dressing or VeraFlo® foam, both available from Kinetic Concepts, Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment, sufficient to maintain a therapeutic pressure at a tissue site. The cover 106 may comprise or consist essentially of a naturally stiff or stretch-resistant polyethylene substrate, film, or foam. In alternative embodiments, the cover 106 may be constructed from Glyptal or pentaphthalic from the Alkyd family of medical grade substrates, films, or foam polymers that polymerize when exposed to oxygen at room temperature. In other embodiments, it may also be comprised or consist essentially of polymers that cross-link upon exposure to body heat, exposure to carbon dioxide, or that evaporate a volatile plasticizer to stiffen, such as substrates, films, or foams made from polyvinyl alcohol.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the therapy system 100, such as the negative-pressure source 104 or the oxygen source 114. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, the oxygen concentration at the tissue interface 108, or the pressure at the tissue interface 108, for example. The controller 110 is also preferably configured to receive one or more input signals, such as a feedback signal, and programmed to modify one or more operating parameters based on the input signals.

Sensors, such as the pressure sensor 120 or the electric sensor 122, are generally known in the art as any apparatus operable to detect or measure a physical phenomenon or property, and generally provide a signal indicative of the phenomenon or property that is detected or measured. For example, the pressure sensor 120 and the electric sensor 122 may be configured to measure one or more operating parameters of the therapy system 100. In some embodiments, the pressure sensor 120 may be a transducer configured to measure pressure in a pneumatic pathway and convert the measurement to a signal indicative of the pressure measured. In some embodiments, for example, the pressure sensor 120 may be a piezoresistive strain gauge. The electric sensor 122 may optionally measure operating parameters of the negative-pressure source 104, such as the voltage or current, in some embodiments. In some embodiments, the oxygen sensor 124 may also provide feedback to the controller 110. Preferably, the signals from the pressure sensor 120, the electric sensor 122, and the oxygen sensor 124 are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

The container 112 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

In operation, the tissue interface 108 may be placed within, over, on, or otherwise proximate to a tissue site. The cover 106 may be placed over the tissue interface 108 and sealed to an attachment surface near the tissue site. For example, the cover 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in container 112. The oxygen source 114 may deliver oxygen, and may increase the pressure in the sealed therapeutic environment to normobaric or hyperbaric levels.

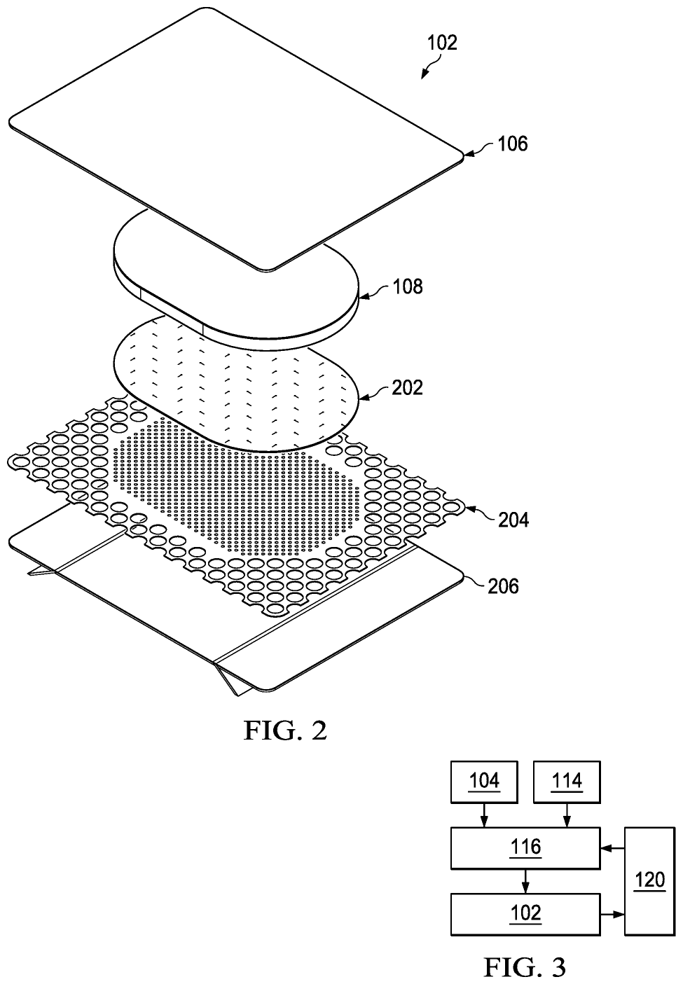

FIG. 2 is a schematic diagram of an example of the dressing 102, illustrating additional details that may be associated with some embodiments. In the example of FIG. 2, the cover 106 generally comprises an inelastic occlusive drape with an adhesive border or layer. The tissue interface 108 of FIG. 2 may comprise a layer of foam filler or bolster, and may additionally or alternatively comprise other layers, such as a comfort layer 202. For example, the comfort layer 202 may comprise or consist essentially of a fenestrated film for reducing or minimizing tissue growth into the layer of foam. Some example embodiments may also comprise a sealing layer 204, which can improve the seal of the dressing 102 and allow it to be repositioned if appropriate. For example, in some embodiments, the sealing layer 204 may comprise a layer of perforated silicone. A release liner 206 may also be included in some embodiments. In some additional embodiments, further fluid-absorbing functionality may be added to the dressing 102 through the incorporation of one or more superabsorbent materials, such as polyacrylates or other materials. For example, the dressing 102 may include an absorbent core made from a Texsus 500 grams per square meter (gsm) superabsorbent textile material, capable of capturing and storing fluids for the duration of therapy. Additional film layers may also be incorporated in the dressing 102 to prevent backflow of fluids between layers of the dressing 102.

The dressing 102 may be assembled in situ, or may be applied as a unit to a tissue site. For example, in some embodiments, the sealing layer 204 may be applied to a tissue site, and then the tissue interface 108 may be applied over the sealing layer 204. The cover 106 may then be applied over the tissue interface 108 and adhered to epidermis around the tissue site. In some embodiments, adhesive from the cover 106 may be pressed through perforations in the sealing layer 204. In other embodiments, the sealing layer 204 may be adhered to the release liner 206, and then the tissue interface 108 coupled to the sealing layer 204. In some embodiments, the cover 106 may also be coupled to the tissue interface 108, so that the tissue interface 108 is disposed between the sealing layer 204 and the cover 106. The release liner 206 may be removed before application to a tissue site.

In use, some embodiments of the therapy system 100 may be operated in one or more modes, depending on the type of therapy desired. For example, an example embodiment of the therapy system 100 may be operated in a first mode for delivery negative-pressure therapy at 2.4 PSI (125 mmHg) of negative pressure. In some embodiments of the therapy system 100 suited for such mode of operation, the dressing 102 may comprise an adhesive-backed, reinforced, flexible but inelastic polyethylene film and a polyurethane foam bolster material. Negative-pressure and/or oxygen therapy may be delivered to the dressing 102 through a dressing interface, or interface pad, that is configured to couple both a dual-lumen tube and a single-lumen tube to the dressing 102. In some embodiments, the dual-lumen tube may be used to deliver negative pressure to the dressing 102 as well as monitor pressure levels within the dressing 102. In some alternative embodiments, the oxygen therapy may be delivered through an outer lumen of a dual-lumen tube, while negative-pressure therapy is administered through an inner lumen of the dual-lumen tube. The therapy system 100 may also be operated in a second mode for delivering hyperbaric oxygen therapy at 0.89 PSI (50 mmHg) to the dressing 102. The additional single-lumen tube may be used for delivering oxygen to the dressing 102 and the tissue site. In some additional or alternative embodiments, the negative-pressure therapy, the normobaric or hyperbaric oxygen therapy, or both, may be delivered to the dressing 102 by another form of flexible conduit, such as a conduit comprising a foam, non-woven material, alternative wicking material, or combination thereof.

FIG. 3 is a simplified flow diagram illustrating additional details that may be associated with some example embodiments of the therapy system 100. In the example embodiment of FIG. 3, the negative-pressure source 104 and the oxygen source 114 may be fluidly coupled to the dressing 102 through the control valve 116, which can control the flow of therapy. The pressure sensor 120 may measure the pressure in the dressing 102, and the measurement can be processed as a feedback signal to operate the control valve 116. Additionally or alternatively, the pressure measurement may be processed as a feedback signal to operate the negative-pressure source 104, the oxygen source 114, or both. Additionally or alternatively, an oxygen sensor may measure the oxygen concentration in the dressing 102, and the measurement can be processed as a feedback signal to operate the negative-pressure source 104, the oxygen source 114, the control valve 116, or some combination thereof.

Some example embodiments of the therapy system 100 may include additional or alternative features, depending on the particular application of negative-pressure and/or oxygen therapy. For example, some embodiments of the dressing 102 may include a cover 106 comprising an elastomeric drape reinforced with polyethylene fiber to form a flexible and customizable cover that can maintain structural integrity during hyperbaric oxygen therapy cycles. In some additional embodiments, the dressing 102 may comprise a self-hardening or self-evaporating polyvinyl alcohol foam adapted to initially form a flexible and customizable cover that can harden to provide a rigid structure for maintaining structural integrity during hyperbaric oxygen therapy cycles. In yet some further embodiments, the dressing 102 may be configured so as to allow for negative-pressure therapy to be applied circumferentially around the tissue site, or around the perimeter edges of the dressing 102, to help maintain the seal around the area of the tissue site during application of normobaric or hyperbaric oxygen therapy treatments.

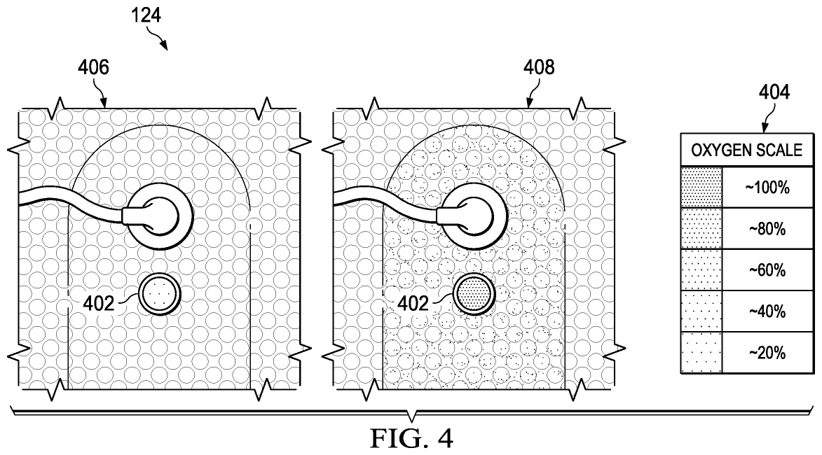

FIG. 4 is a schematic diagram of another example of a portion of the dressing 102, illustrating additional details that may be associated with embodiments of the oxygen sensor 124. The oxygen sensor 124 comprises a colorimetric oxygen-sensing indicator 402, and a corresponding colorimetric scale 404 indicative of oxygen concentration. The oxygen-sensing indicator 402 may be configured to detect and indicate the presence or concentration of oxygen. For example, in some embodiments, the oxygen-sensing indicator 402 may be a layer in the dressing 102, comprising or consisting essentially of a composition of a layered silicate, a cationic surfactant, an organic colorant, and a reducing agent, such as described in U.S. Pat. No. 6,703,245. Other suitable compositions may be similar to oxygen-indicating tablets manufactured by Impak Corporation. In some additional or alternative embodiments, the oxygen-sensing indicator 402 may be coated on an occlusive layer, such as the drape or cover, of the dressing 102. For example, the oxygen-sensing indicator 402 may be a colorimetric change media chemically applied or pattern-coated or mechanically bound or coated to an adhesive or film of the occlusive layer of the dressing 102. The oxygen sensor 124 may be configured to react at a threshold concentration in some embodiments. For example, in the example of FIG. 4, the oxygen sensor 124 is configured to change color if the oxygen concentration exceeds 20%. As shown in the first view 406 of FIG. 4, the oxygen-sensing indicator 402 may initially appear as a first color before exposure to oxygen therapy, and may appear as a second color following a period of exposure to oxygen therapy, as shown in the second view 408 of FIG. 4.

In some embodiments, the oxygen sensor 124 may be comprised of solutions containing laboratory-grade redox reaction dye indicators (such as Methylene blue or AlamarBlue) that are essentially clear in the absence of oxygen but turn blue in the presence of oxygen. Their formulation may be adjusted or oxygen scavengers may be added such that the color change (blue) begins at oxygen concentrations above ˜21%, and concentrations below the threshold do not trigger a colorimetric response. The color change may be reversed with the addition of glucose (dextrose).

Color changes may be varied in other embodiments. For example, Phenosafranine can be used for a solution that turns red when oxygen is introduced. Phenosafranine can also be mixed with Methylene blue to form a solution that turns pink in the presence of oxygen. Indigo carmine gives a solution that can change from yellow to green as oxygen is introduced. Use of Resazurin can render a solution that changes from pale blue to a purple-pink in the presence of oxygen.

The colorimetric scale 404 may be associated with the oxygen sensor 124 in various ways. For example, the colorimetric scale 404 may be printed, adhered, or otherwise disposed on top of the cover 106 in some embodiments. As illustrated in the example of FIG. 4, the colorimetric scale 404 may comprise a group, family, or system of colors graduated in scale signifying a different oxygen concentration or therapy level. For example, a pink color may be indicative of 20% oxygen concentration, red may be indicative of 40% oxygen concentration, purple may be indicative of 60% oxygen concentration, and blue may indicate 80% oxygen concentration. A covering may enclose the scale and prevent or minimize color distortion.

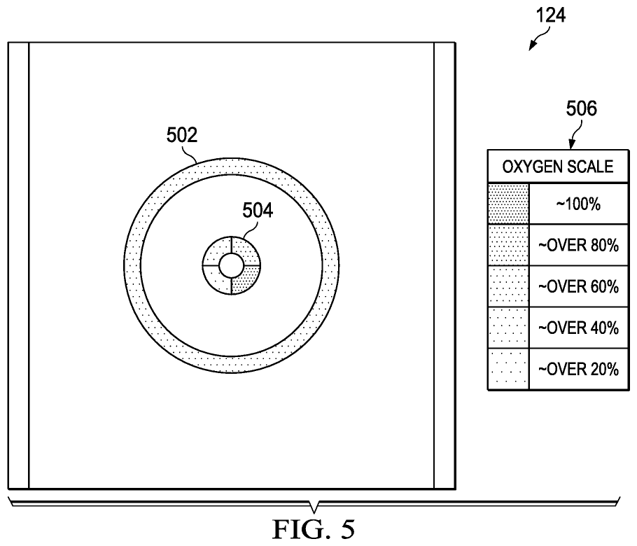

FIG. 5 is a schematic diagram of another example embodiment of a portion of the dressing 102, illustrating additional details that may be associated with some embodiments of the oxygen sensor 124. In the example of FIG. 5, the oxygen sensor 124 may comprise an oxygen concentration indicator ring 502, and an oxygen concentration scale 504 disposed within the oxygen concentration indicator ring 502. In some instances, an additional separate legend 506 may also be included for a user to reference.

The systems, apparatuses, and methods described herein may provide significant advantages. For example, the therapy system 100 may be used to combine topical negative-pressure therapy with oxygen therapy, and maximize the exposure of a tissue site to oxygen-rich therapy. Further, by alternating the delivery of negative-pressure wound therapy with topical oxygen therapy, the negative pressure may beneficially act as a delivery vehicle for the oxygen to be drawn in more direct contact with the surface of a tissue site, such as a wound bed, as opposed to only a passive introduction of oxygen in a general vicinity of a tissue site, as may be the case with most topical oxygen therapy systems currently available. Accordingly, the advantages of negative-pressure wound therapy, such as managing wound margins, fluid removal, and providing a sterile barrier to infection, may be combined with the benefits of providing an oxygen-rich environment to a tissue site to provide an overall wound-management solution. The oxygen therapy may be hypobaric, normobaric, hyperbaric, or any combination thereof, and may be continuous or intermittent, without the need for confining a patient in a costly, specialized chamber. An oxygen indicator such as the oxygen sensor 124 may also facilitate the indication of the presence or relative concentration of oxygen in a single, occlusive dressing, without additional power requirements. The incorporation of the oxygen indicator may allow for clinicians to more easily discern the presence and concentration or level of oxygen at a tissue site, which otherwise may not be possible since oxygen is odorless, appears colorless to the naked eye, and thus offers no discernable difference between oxygen and normal air.

While shown in a few illustrative embodiments, a person having ordinary skill in the art will recognize that the systems, apparatuses, and methods described herein are susceptible to various changes and modifications. Features and elements described in the context of one example embodiment may be combined with features and elements of other example embodiments. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. An apparatus for providing negative-pressure therapy with oxygen to a tissue site, the apparatus comprising:
a tissue interface configured to transport fluid to the tissue site; and
a cover configured to provide a fluid seal around the tissue interface, the cover comprising a polyethylene substrate, film, or foam.

2. An apparatus for providing negative-pressure therapy with oxygen to a tissue site, the apparatus comprising:
a tissue interface configured to transport fluid to the tissue site; and
a cover configured to provide a fluid seal around the tissue interface, the cover comprising a Glyptal or pentaphthalic substrate, film, or foam.

3. An apparatus for providing negative-pressure therapy with oxygen to a tissue site, the apparatus comprising:
a tissue interface configured to transport fluid to the tissue site; and
a cover configured to provide a fluid seal around the tissue interface, the cover comprising a polymer that cross-links when exposed to oxygen at room temperature.

4. An apparatus for providing negative-pressure therapy with oxygen to a tissue site, the apparatus comprising:
a tissue interface configured to transport fluid to the tissue site; and
a cover configured to provide a fluid seal around the tissue interface, the cover comprising a polymer that cross-links when exposed to body heat or carbon dioxide.

5. An apparatus for providing negative-pressure therapy with oxygen to a tissue site, the apparatus comprising:
a tissue interface configured to transport fluid to the tissue site; and
a cover configured to provide a fluid seal around the tissue interface, the cover comprising a polymer that evaporates a volatile plasticizer to increase rigidity.

6. The apparatus of any of claims 1-5, wherein the oxygen is hyperbaric.

7. The apparatus of any of claims 1-6, further comprising an oxygen indicator coupled to the tissue interface.

8. The apparatus of claim 7, wherein the oxygen indicator is configured to react to oxygen concentrations in the tissue interface that exceed a threshold.

9. The apparatus of claim 8, wherein the threshold is at least 20% oxygen concentration.

10. The apparatus of claim 8, wherein the reaction is reversible.

11. The apparatus of any of claims 8-10, wherein the reaction is a colorimetric reaction.

12. The apparatus of any preceding claim, further comprising:
a negative-pressure source fluidly coupled to the tissue interface; and
an oxygen source fluidly coupled to the tissue interface.

13. The apparatus of claim 12, further comprising:
a pressure sensor configured to measure pressure at the tissue interface; and
a controller configured to operate the oxygen source based on a signal from the pressure sensor indicative of the pressure measured at the tissue interface.

14. The apparatus of claim 12, further comprising:
an oxygen sensor configured to measure oxygen concentration at the tissue interface; and
a controller configured to operate at least one of the negative-pressure source and the oxygen source based on a signal from the oxygen sensor indicative of the oxygen concentration measured at the tissue interface.

15. The apparatus of claim 12, further comprising:
a pressure sensor configured to measure pressure at the tissue interface;
an oxygen sensor configured to measure oxygen concentration at the tissue interface; and
a controller configured to operate at least one of the negative-pressure source and the oxygen source based on at least one of a signal from the pressure sensor or the oxygen sensor.

16. An apparatus for providing negative-pressure therapy with oxygen to a tissue site, the apparatus comprising:
a tissue interface configured to transport fluid to the tissue site;
an occlusive cover configured to provide a fluid seal around the tissue interface; and
an oxygen indicator coupled to the tissue interface.

17. The apparatus of claim 16, wherein the oxygen indicator is configured to react to oxygen concentrations in the tissue interface that exceed a threshold.

18. The apparatus of claim 17, wherein the threshold is at least 20% oxygen concentration.

19. The apparatus of claim 17 or claim 18, wherein the reaction is reversible.

20. The apparatus of any of claims 17-19, wherein the reaction is a colorimetric reaction.

21. A method of providing therapy to a tissue site, the method comprising:
applying a tissue interface to the tissue site;
applying a cover over the tissue interface;
sealing the cover around the tissue interface; and
selectively providing negative pressure and oxygen to the tissue interface.

22. The method of claim 21, wherein the oxygen is hypobaric oxygen.

23. The method of claim 21, wherein the oxygen is negatively pressurized between −25 mmHg and −200 mmHg.

24. The method of claim 21, wherein the oxygen is normobaric oxygen or hyperbaric oxygen.

25. The method of claim 21, wherein the oxygen is positively pressurized between 5 mmHg and 50 mmHg.

26. The method of claim 21, wherein the oxygen is positively pressurized between 1 atm and 3 atm.

27. The method of any of claims 21-26, wherein the oxygen is at a concentration of between 50% to 100%.

28. The systems, apparatuses, and methods substantially as described herein.
